SEQUENCE LISTING (32 sequences)

SEQ ID NO: 1 - protein, 3075 aa, Homo sapiens
MRGGVLLVLLLCVAAQCRQRGLFPAILNLASNAHISTNATCGEKGPEMFCKLVEHVPGRPVRNPQCRICDGNSANPRERHPISHAIDGTNNWWQSPSIQNGREYHWVTITLDLRQVFQVAYVIIKAANAPRPGNWILERSLDGTTFSPWQYYAVSDSECLSRYNITPRRGPPTYRADDEVICTSYYSRLVPLEHGEIHTSLINGRPSADDLSPKLLEFTSARYIRLRLQRIRTLNADLMTLSHREPKELDPIVTRRYYYSIKDISVGGMCICYGHASSCPWDETTKKLQCQCEHNTCGESCNRCCPGYHQQPWRPGTVSSGNTCEACNCHNKAKDCYYDESVAKQKKSLNTAGQFRGGGVCINCLQNTMGINCETCIDGYYRPHKVSPYEDEPCRPCNCDPVGSLSSVCIKDDLHSDLHNGKQPGQCPCKEGYTGEKCDRCQLGYKDYPTCVSCGCNPVGSASDEPCTGPCVCKENVEGKACDRCKPGFYNLKEKNPRGCSECFCFGVSDVCSSLSWPVGQVNSMSGWLVTDLISPRKIPSQQDALGGRHQVSINNTAVMQRLAPKYYWAAPEAYLGNKLTAFGGFLKYTVSYDIPVETVDSNLMSHADVIIKGNGLTLSTQAEGLSLQPYEEYLNVVRLVPENFQDFHSKRQIDRDQLMTVLANVTHLLIRANYNSAKMALYRLESVSLDIASSNAIDLVVAADVEHCECPQGYTGTSCESCLSGYYRVDGILFGGICQPCECHGHAAECNVHGVCIACAHNTTGVHCEQCLPGFYGEPSRGTPGDCQPCACPLTIASNNFSPTCHLNDGDEVVCDWCAPGYSGAWCERCADGYYGNPTVPGESCVPCDCSGNVDPSEAGHCDSVTGECLKCLGNTDGAHCERCADGFYGDAVTAKNCRACECHVKGSHSAVCHLETGLCDCKPNVTGQQCDQCLHGYYGLDSGHGCRPCNCSVAGSVSDGCTDEGQCHCVPGVAGKRCDRCAHGFYAYQDGSCTPCDCPHTQNTCDPETGECVCPPHTQGVKCEECEDGHWGYDAEVGCQACNCSLVGSTHHRCDVVTGHCQCKSKFGGRACDQCSLGYRDFPDCVPCDCDLRGTSGDACNLEQGLCGCVEETGACPCKENVFGPQCNECREGTFALRADNPLGCSPCFCSGLSHLCSELEDYVRTPVTLGSDQPLLRVVSQSNLRGTTEGVYYQAPDFLLDAATVRQHIRAEPFYWRLPQQFQGDQLMAYGGKLKYSVAFYSLDGVGTSNFEPQVLIKGGRIRKQVIYMDAPAPENGVRQEQEVAMRENFWKYFNSVSEKPVTREDFMSVLSDIEYILIKASYGQGLQQSRISDISMEVGRKAEKLHPEEEVASLLENCVCPPGTVGFSCQDCAPGYHRGKLPAGSDRGPRPLVAPCVPCSCNNHSDTCDPNTGKCLNCGDNTAGDHCDVCTSGYYGKVTGSASDCALCACPHSPPASFSPTCVLEGDHDFRCDACLLGYEGKHCERCSSSYYGNPQTPGGSCQKCDCNPHGSVHGDCDRTSGQCVCRLGASGLRCDECEPRHILMETDCVSCDDECVGVLLNDLDEIGDAVLSLNLTGIIPVPYGILSNLENTTKYLQESLLKENMQKDLGKIKLEGVAEETDNLQKKLTRMLASTQKVNRATERIFKESQDLAIAIERLQMSITEIMEKTTLNQTLDEDFLLPNSTLQNMQQNGTSLLEIMQIRDFTQLHQNATLELKAAEDLLSQIQENYQKPLEELEVLKEAASHVLSKHNNELKAAEALVREAEAKMQESNHLLLMVNANLREFSDKKLHVQEEQNLTSELIVQGRGLIDAAAAQTDAVQDALEHLEDHQDKLLLWSAKIRHHIDDLVMHMSQRNAVDLVYRAEDHAAEFQRLADVLYSGLENIRNVSLNATSAAYVHYNIQSLIEESEELARDAHRTVTETSLLSESLVSNGKAAVQRSSRFLKEGNNLSRKLPGIALELSELRNKTNRFQENAVEITRQTNESLLILRAIPKGIRDKGAKTKELATSASQSAVSTLRDVAGLSQELLNTSASLSRVNTTLRETHQLLQDSTMATLLAGRKVKDVEIQANLLFDRLKPLKMLEENLSRNLSEIKLLISQARKQAASIKVAVSADRDCIRAYQPQISSTNYNTLTLNVKTQEPDNLLFYLGSSTASDFLAVEMRRGRVAFLWDLGSGSTRLEFPDFPIDDNRWHSIHVARFGNIGSLSVKEMSSNQKSPTKTSKSPGTANVLDVNNSTLMFVGGLGGQIKKSPAVKVTHFKGCLGEAFLNGKSIGLWNYIEREGKCRGCFGSSQNEDPSFHFDGSGYSVVEKSLPATVTQIIMLFNTFSPNGLLLYLGSYGTKDFLSIELFRGRVKVMTDLGSGPITLLTDRRYNNGTWYKIAFQRNRKQGVLAVIDAYNTSNKETKQGETPGASSDLNRLDKDPIYVGGLPRSRVVRRGVTTKSFVGCIKNLEISRSTFDLLRNSYGVRKGCLLEPIRSVSFLKGGYIELPPKSLSPESEWLVTFATTNSSGIILAALGGDVEKRGDREEAHVPFFSVMLIGGNIEVHVNPGDGTGLRKALLHAPTGTCSDGQAHSISLVRNRRIITVQLDENNPVEMKLGTLVESRTINVSNLYVGGIPEGEGTSLLTMRRSFHGCIKNLIFNLELLDFNSAVGHEQVDLDTCWLSERPKLAPDAEDSKLLPEPRAFPEQCVVDAALEYVPGAHQFGLTQNSHFILPFNQSAVRKKLSVELSIRTFASSGLIYYMAHQNQADYAVLQLHGGRLHFMFDLGKGRTKVSHPALLSDGKWHTVKTDYVKRKGFITVDGRESPMVTVVGDGTMLDVEGLFYLGGLPSQYQARKIGNITHSIPACIGDVTVNSKQLDKDSPVSAFTVNRCYAVAQEGTYFDGSGYAALVKEGYKVQSDVNITLEFRTSSQNGVLLGISTAKVDAIGLELVDGKVLFHVNNGAGRITAAYEPKTATVLCDGKWHTLQANKSKHRITLIVDGNAVGAESPHTQSTSVDTNNPIYVGGYPAGVKQKCLRSQTSFRGCLRKLALIKSPQVQSFDFSRAFELHGVFLHSCPGTES

SEQ ID NO: 2 - protein, 1786 aa, Homo sapiens
MGLLQLLAFSFLALCRARVRAQEPEFSYGCAEGSCYPATGDLLIGRAQKLSVTSTCGLHKPEPYCIVSHLQEDKKCFICNSQDPYHETLNPDSHLIENVVTTFAPNRLKIWWQSENGVENVTIQLDLEAEFHFTHLIMTFKTFRPAAMLIERSSDFGKTWGVYRYFAYDCEASFPGISTGPMKKVDDIICDSRYSDIEPSTEGEVIFRALDPAFKIEDPYSPRIQNLLKITNLRIKFVKLHTLGDNLLDSRMEIREKYYYAVYDMVVRGNCFCYGHASECAPVDGFNEEVEGMVHGHCMCRHNTKGLNCELCMDFYHDLPWRPAEGRNSNACKKCNCNEHSISCHFDMAVYLATGNVSGGVCDDCQHNTMGRNCEQCKPFYYQHPERDIRDPNFCERCTCDPAGSQNEGICDSYTDFSTGLIAGQCRCKLNVEGEHCDVCKEGFYDLSSEDPFGCKSCACNPLGTIPGGNPCDSETGHCYCKRLVTGQHCDQCLPEHWGLSNDLDGCRPCDCDLGGALNNSCFAESGQCSCRPHMIGRQCNEVEPGYYFATLDHYLYEAEEANLGPGVSIVERQYIQDRIPSWTGAGFVRVPEGAYLEFFIDNIPYSMEYDILIRYEPQLPDHWEKAVITVQRPGRIPTSSRCGNTIPDDDNQVVSLSPGSRYVVLPRPVCFEKGTNYTVRLELPQYTSSDSDVESPYTLIDSLVLMPYCKSLDIFTVGGSGDGVVTNSAWETFQRYRCLENSRSVVKTPMTDVCRNIIFSISALLHQTGLACECDPQGSLSSVCDPNGGQCQCRPNVVGRTCNRCAPGTFGFGPSGCKPCECHLQGSVNAFCNPVTGQCHCFQGVYARQCDRCLPGHWGFPSCQPCQCNGHADDCDPVTGECLNCQDYTMGHNCERCLAGYYGDPIIGSGDHCRPCPCPDGPDSGRQFARSCYQDPVTLQLACVCDPGYIGSRCDDCASGYFGNPSEVGGSCQPCQCHNNIDTTDPEACDKETGRCLKCLYHTEGEHCQFCRFGYYGDALRQDCRKCVCNYLGTVQEHCNGSDCQCDKATGQCLCLPNVIGQNCDRCAPNTWQLASGTGCDPCNCNAAHSFGPSCNEFTGQCQCMPGFGGRTCSECQELFWGDPDVECRACDCDPRGIETPQCDQSTGQCVCVEGVEGPRCDKCTRGYSGVFPDCTPCHQCFALWDVIIAELTNRTHRFLEKAKALKISGVIGPYRETVDSVERKVSEIKDILAQSPAAEPLKNIGNLFEEAEKLIKDVTEMMAQVEVKLSDTTSQSNSTAKELDSLQTEAESLDNTVKELAEQLEFIKNSDIRGALDSITKYFQMSLEAEERVNASTTEPNSTVEQSALMRDRVEDVMMERESQFKEKQEEQARLLDELAGKLQSLDLSAAAEMTCGTPPGASCSETECGGPNCRTDEGERKCGGPGCGGLVTVAHNAWQKAMDLDQDVLSALAEVEQLSKMVSEAKLRADEAKQSAEDILLKTNATKEKMDKSNEELRNLIKQIRNFLTQDSADLDSIEAVANEVLKMEMPSTPQQLQNLTEDIRERVESLSQVEVILQHSAADIARAEMLLEEAKRASKSATDVKVTADMVKEALEEAEKAQVAAEKAIKQADEDIQGTQNLLTSIESETAASEETLFNASQRISELERNVEELKRKAAQNSGEAEYIEKVVYTVKQSAEDVKKTLDGELDEKYKKVENLIAKKTEESADARRKAEMLQNEAKTLLAQANSKLQLLKDLERKYEDNQRYLEDKAQELARLEGEVRSLLKDISQKVAVYSTCL

SEQ ID NO: 3 - protein, 1609 aa, Homo sapiens
MRGSHRAAPALRPRGRLWPVLAVLAAAAAAGCAQAAMDECTDEGGRPQRCMPEFVNAAFNVTVVATNTCGTPPEEYCVQTGVTGVTKSCHLCDAGQPHLQHGAAFLTDYNNQADTTWWQSQTMLAGVQYPSSINLTLHLGKAFDITYVRLKFHTSRPESFAIYKRTREDGPWIPYQYYSGSCENTYSKANRGFIRTGGDEQQALCTDEFSDISPLTGGNVAFSTLEGRPSAYNFDNSPVLQEWVTATDIRVTLNRLNTFGDEVFNDPKVLKSYYYAISDFAVGGRCKCNGHASECMKNEFDKLVCNCKHNTYGVDCEKCLPFFNDRPWRRATAESASECLPCDCNGRSQECYFDPELYRSTGHGGHCTNCQDNTDGAHCERCRENFFRLGNNEACSSCHCSPVGSLSTQCDSYGRCSCKPGVMGDKCDRCQPGFHSLTEAGCRPCSCDPSGSIDECNIETGRCVCKDNVEGFNCERCKPGFFNLESSNPRGCTPCFCFGHSSVCTNAVGYSVYSISSTFQIDEDGWRAEQRDGSEASLEWSSERQDIAVISDSYFPRYFIAPAKFLGKQVLSYGQNLSFSFRVDRRDTRLSAEDLVLEGAGLRVSVPLIAQGNSYPSETTVKYVFRLHEATDYPWRPALTPFEFQKLLNNLTSIKIRGTYSERSAGYLDDVTLASARPGPGVPATWVESCTCPVGYGGQFCEMCLSGYRRETPNLGPYSPCVLCACNGHSETCDPETGVCNCRDNTAGPHCEKCSDGYYGDSTAGTSSDCQPCPCPGGSSCAVVPKTKEVVCTNCPTGTTGKRCELCDDGYFGDPLGRNGPVRLCRLCQCSDNIDPNAVGNCNRLTGECLKCIYNTAGFYCDRCKDGFFGNPLAPNPADKCKACNCNLYGTMKQQSSCNPVTGQCECLPHVTGQDCGACDPGFYNLQSGQGCERCDCHALGSTNGQCDIRTGQCECQPGITGQHCERCEVNHFGFGPEGCKPCDCHPEGSLSLQCKDDGRCECREGFVGNRCDQCEENYFYNRSWPGCQECPACYRLVKDKVADHRVKLQELESLIANLGTGDEMVTDQAFEDRLKEAEREVMDLLREAQDVKDVDQNLMDRLQRVNNTLSSQISRLQNIRNTIEETGNLAEQARAHVENTERLIEIASRELEKAKVAAANVSVTQPESTGDPNNMTLLAEEARKLAERHKQEADDIVRVAKTANDTSTEAYNLLLRTLAGENQTAFEIEELNRKYEQAKNISQDLEKQAARVHEEAKRAGDKAVEIYASVAQLSPLDSETLENEANNIKMEAENLEQLIDQKLKDYEDLREDMRGKELEVKNLLEKGKTEQQTADQLLARADAAKALAEEAAKKGRDTLQEANDILNNLKDFDRRVNDNKTAAEEALRKIPAINQTITEANEKTREAQQALGSAAADATEAKNKAHEAERIASAVQKNATSTKAEAERTFAEVTDLDNEVNNMLKQLQEAEKELKRKQDDADQDMMMAGMASQAAQEAEINARKAKNSVTSLLSIINDLLEQLGQLDTVDLNKLNEIEGTLNKAKDEMKVSDLDRKVSDLENEAKKQEAAIMDYNRDIEEIMKDIRNLEDIRKTLPSGCFNTPSIEKP

SEQ ID NO: 4 - protein, 1464 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 5 - protein, 1366 aa, Homo sapiens [sequence omitted]

SEQ ID NO: 6 - protein, 1466 aa, Homo sapiens
MMSFVQKGSWLLLALLHPTIILAQQEAVEGGCSHLGQSYADRDVWKPEPCQICVCDSGSVLCDDIICDDQELDCPNPEIPFGECCAVCPQPPTAPTRPPNGQGPQGPKGDPGPPGIPGRNGDPGIPGQPGSPGSPGPPGICESCPTGPQNYSPQYDSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPGLAGAPGLRGGAGPPGPEGGKGAAGPPGPPGAAGTPGLQGMPGERGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGFAPYYGDEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVHLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 7 - protein, 2201 aa, Homo sapiens
MGAMTQLLAGVFLAFLALATEGGVLKKVIRHKRQSGVNATLPEENQPVVFNHVYNIKLPVGSQCSVDLESASGEKDLAPPSEPSESFQEHTVDGENQIVFTHRINIPRRACGCAAAPDVKELLSRLEELENLVSSLREQCTAGAGCCLQPATGRLDTRPFCSGRGNFSTEGCGCVCEPGWKGPNCSEPECPGNCHLRGRCIDGQCICDDGFTGEDCSQLACPSDCNDQGKCVNGVCICFEGYAGADCSREICPVPCSEEHGTCVDGLCVCHDGFAGDDCNKPLCLNNCYNRGRCVENECVCDEGFTGEDCSELICPNDCFDRGRCINGTCYCEEGFTGEDCGKPTCPHACHTQGRCEEGQCVCDEGFAGLDCSEKRCPADCHNRGRCVDGRCECDDGFTGADCGELKCPNGCSGHGRCVNGQCVCDEGYTGEDCSQLRCPNDCHSRGRCVEGKCVCEQGFKGYDCSDMSCPNDCHQHGRCVNGMCVCDDGYTGEDCRDRQCPRDCSNRGLCVDGQCVCEDGFTGPDCAELSCPNDCHGQGRCVNGQCVCHEGFMGKDCKEQRCPSDCHGQGRCVDGQCICHEGFTGLDCGQHSCPSDCNNLGQCVSGRCICNEGYSGEDCSEVSPPKDLVVTEVTEETVNLAWDNEMRVTEYLVVYTPTHEGGLEMQFRVPGDQTSTIIQELEPGVEYFIRVFAILENKKSIPVSARVATYLPAPEGLKFKSIKETSVEVEWDPLDIAFETWEIIFRNMNKEDEGEITKSLRRPETSYRQTGLAPGQEYEISLHIVKNNTRGPGLKRVTTTRLDAPSQIEVKDVTDTTALITWFKPLAEIDGIELTYGIKDVPGDRTTIDLTEDENQYSIGNLKPDTEYEVSLISRRGDMSSNPAKETFTTGLDAPRNLRRVSQTDNSITLEWRNGKAAIDSYRIKYAPISGGDHAEVDVPKSQQATTKTTLTGLRPGTEYGIGVSAVKEDKESNPATINAATELDTPKDLQVSETAETSLTLLWKTPLAKFDRYRLNYSLPTGQWVGVQLPRNTTSYVLRGLEPGQEYNVLLTAEKGRHKSKPARVKASTEQAPELENLTVTEVGWDGLRLNWTAADQAYEHFIIQVQEANKVEAARNLTVPGSLRAVDIPGLKAATPYTVSIYGVIQGYRTPVLSAEASTGETPNLGEVVVAEVGWDALKLNWTAPEGAYEYFFIQVQEADTVEAAQNLTVPGGLRSTDLPGLKAATHYTITIRGVTQDFSTTPLSVEVLTEEVPDMGNLTVTEVSWDALRLNWTTPDGTYDQFTIQVQEADQVEEAHNLTVPGSLRSMEIPGLRAGTPYTVTLHGEVRGHSTRPLAVEVVTEDLPQLGDLAVSEVGWDGLRLNWTAADNAYEHFVIQVQEVNKVEAAQNLTLPGSLRAVDIPGLEAATPYRVSIYGVIRGYRTPVLSAEASTAKEPEIGNLNVSDITPESFNLSWMATDGIFETFTIEIIDSNRLLETVEYNISGAERTAHISGLPPSTDFIVYLSGLAPSIRTKTISATATTEALPLLENLTISDINPYGFTVSWMASENAFDSFLVTVVDSGKLLDPQEFTLSGTQRKLELRGLITGIGYEVMVSGFTQGHQTKPLRAEIVTEAEPEVDNLLVSDATPDGFRLSWTADEGVFDNFVLKIRDTKKQSEPLEITLLAPERTRDLTGLREATEYEIELYGISKGRRSQTVSAIATTAMGSPKEVIFSDITENSATVSWRAPTAQVESFRITYVPITGGTPSMVTVDGTKTQTRLVKLIPGVEYLVSIIAMKGFEESEPVSGSFTTALDGPSGLVTANITDSEALARWQPAIATVDSYVISYTGEKVPEITRTVSGNTVEYALTDLEPATEYTLRIFAEKGPQKSSTITAKFTTDLDSPRDLTATEVQSETALLTWRPPRASVTGYLLVYESVDGTVKEVIVGPDTTSYSLADLSPSTHYTAKIQALNGPLRSNMIQTIFTTIGLLYPFPKDCSQAMLNGDTTSGLYTIYLNGDKAQALEVFCDMTSDGGGWIVFLRRKNGRENFYQNWKAYAAGFGDRREEFWLGLDNLNKITAQGQYELRVDLRDHGETAFAVYDKFSVGDAKTRYKLKVEGYSGTAGDSMAYHNGRSFSTFDKDTDSAITNCALSYKGAFWYRNCHRVNLMGRYGDNNHSQGVNWFHWKGHEHSIQFAEMKLRPSNFRNLEGRRKRA

SEQ ID NO: 8 - protein, 6 aa, Artificial
  Synthetic peptide
GFXGER

SEQ ID NO: 9 - protein, 13 aa, Homo sapiens
GEFYFDLRLKGDK

SEQ ID NO: 10 - protein, 3 aa, Homo sapiens
LRE

SEQ ID NO: 11 - protein, 13 aa, Homo sapiens
AASIKAVAVSADR

SEQ ID NO: 12 - protein, 12 aa, Homo sapiens
LAIKNDNLVYVY

SEQ ID NO: 13 - protein, 13 aa, Homo sapiens
RYVVLPRPVLFEK

SEQ ID NO: 14 - protein, 19 aa, Homo sapiens
ELTGAARKGSGRRLVKGPD

SEQ ID NO: 15 - protein, 17 aa, Homo sapiens
MKKTRGTLLALERKDHS

SEQ ID NO: 16 - protein, 7 aa, Homo sapiens
SVVYGLR

SEQ ID NO: 17 - protein, 4 aa, Homo sapiens
YIIR

SEQ ID NO: 18 - protein, 4 aa, homo sapiens
GSKS

SEQ ID NO: 19 - protein, 11 aa, Homo sapiens
TYSSPEDGIHE

SEQ ID NO: 20 - protein, 11 aa, Homo sapiens
WQPPRARITGY

SEQ ID NO: 21 - protein, 4 aa, Homo sapiens
LREA

SEQ ID NO: 22 - protein, 6 aa, Homo sapiens
  MOD_RES (6)..(6): Orn
GRGDSX

SEQ ID NO: 23 - protein, 12 aa, Homo sapiens
DVISLYNFKHIY

SEQ ID NO: 24 - protein, 11 aa, Homo sapiens
DRVPHSRNSIT

SEQ ID NO: 25 - protein, 6 aa, Homo sapiens
KREDVY

SEQ ID NO: 26 - protein, 6 aa, Homo sapiens
KREDVY

SEQ ID NO: 27 - protein, 5 aa, Homo sapiens
EILDV

SEQ ID NO: 28 - protein, 5 aa, Homo sapiens
EILDV

SEQ ID NO: 29 - protein, 20 aa, Homo sapiens
WTPPRAQITGYRLTVGLTRR

SEQ ID NO: 30 - protein, 5 aa, Homo sapiens
YIIRA

SEQ ID NO: 31 - protein, 5 aa, Homo sapiens
KRSRA

SEQ ID NO: 32 - protein, 25 aa, Homo sapiens
DELPQLVTLPHPNLHGPEILDVPST

(19) United States
(12) Patent Application Publication
(10) Pub. No.: US 2013/0156819 A1
(43) Pub. Date: Jun. 20, 2013

(54) ISOLATION OF MESENCHYMAL STEM CELLS

(71) Applicants: Eberhard-Karls-Universit°t Tübingen Universit°tskl, Tübingen (DE); Baden-Württemberg Stiftung gGmbH, Stuttgart (DE)

(72) Inventors: Wilhelm Aicher, Ammerbuch (DE); Brigitte Angres, Pfullingen (DE)

(73) Assignees: Baden-Württemberg Stiftung gGmbH, Stuttgart (DE); Eberhard-Karls-Universit°t Tübingen Universit°tsklinikum, Tübingen (DE)

(21) Appl. No.: 13/706,308

(22) Filed: Dec. 5, 2012

Related U.S. Application Data
(63) Continuation of application No. PCT/EP2011/059393, filed on Jun. 7, 2011.

(30) Foreign Application Priority Data
Jun. 7, 2010 (DE) 102010023837.6

(51) Int. Cl. C07K 7/06; A61K 35/28; C07K 7/08; A61L 31/16; C07K 5/083; C07K 5/103; C07K 14/47; A61L 31/08; C12N 5/071; C07K 5/107

(52) U.S. Cl. CPC C07K 7/06; C12N 5/0668; A61K 35/28; C07K 7/08; C07K 5/1016; C07K 5/0808; C07K 5/1008; C07K 5/101; C07K 14/47; A61L 31/08; A61L 31/16
USPC 424/400; 435/325; 435/7.1; 435/377; 514/16.7; 514/17.1; 514/17.2; 514/20.9; 514/13.8; 514/9.4; 514/7.6; 514/9.3; 424/93.7; 530/329; 530/327; 530/326; 530/330; 530/325; 530/331; 530/328

(57) ABSTRACT

The present invention relates to peptides or fragments thereof, which peptides bind to mesenchymal stem cells. The present invention also relates to a method for identifying, isolating, specifically selecting and/or enriching mesenchymal stem cells, wherein the peptides, or fragments thereof, are employed for specifically binding to the mesenchymal stem cells. Also, the present invention relates to the use of the peptides of the invention, or fragments thereof, and of the mesenchymal stem cells isolated with the peptides of the invention, or fragments thereof, for treating, injuries and/or degenerated bone, cartilage or tissues.

### CROSS-REFERENCES TO RELATED APPLICATIONS

This application is a continuation of international patent application PCT/EP2011/059393, filed on Jun. 7, 2011 designating the U.S., which international patent application has been published in German and claims priority to German patent application DE 10 2010 023 837.6, filed on Jun. 7, 2010. The entire contents of these priority applications are incorporated herein by reference.

### BACKGROUND OF THE INVENTION

The present invention relates, inter alia, to the isolation, identification and/or activation of mesenchymal stem cells with proteins or peptides derived therefrom.

Mesenchymal stem cells (MSCs) are pluripotent cells and can, in suitable in vitro and in vivo conditions, differentiate into various mesenchymal tissues, for example into bone tissue, fat tissue, muscles, cartilage. MSCs have the property of adhering stably and rapidly to plastic or glass surfaces, and they have a fibroblastoid phenotype. MSCs are easily distinguishable from hematopoietic stem cells, as they do not express any specific hematopoietic surface markers. However, still no specific surface antigen for MSCs is known in the prior art; the surface molecules expressed by them are also to be found on surfaces of endothelial, mesenchymal and epithelial cells, and muscle cells.

The term “mesenchymal stem cell” (MSC) is not defined completely uniformly in the literature. Basically two cell types can be distinguished: MSCs that are isolated directly from non-hematopoietic primary tissue (e.g. bone marrow, fat tissue, placenta) and cells that are cultivated, and that differentiate in culture from these primary cells into adherent, fibroblastoid cells. There they express cell surface markers such as CD29, CD44, CD73, CD90, CD105, CD166, but are negative for the hematopoietic stem cell marker CD34 and the pan-leukocyte marker CD45. Generally the cells generated in culture are called mesenchymal stem cells, because even after this process they still possess multipotent differentiation capacity.

Owing to their multipotency, i.e. their property of being able to differentiate, under suitable in vitro and in vivo conditions, into various mesenchymal tissues (for example bone, fat, muscle, cartilage, etc.), mesenchymal stem cells are already used for therapeutic purposes. For example, differentiable MSCs isolated from placenta, bone marrow and fat tissue, expanded in vitro, can be differentiated into osteoblasts, chondrocytes and myocytes, and can then be used again in vivo, for example for the regeneration of bone, cartilage, tendons, muscles, fat tissue and stroma.

In the state of the art, on the one hand, mesenchymal stem cells can be isolated from bone marrow by means of antibodies that are directed against the low-affinity nerve growth factor receptor CD271 (Quirici et al., “Isolation of bone marrow mesenchymal stem cells by anti-nerve growth factor receptor antibodies”, Exp. Hematol., 2002, 30(7): 783-791). It has also been described that MSCs can be isolated by means of antibodies to SH2 (CD105), SH3 (CD73) and SH4 (CD73) (see Barry F, et al., “The SH-3 and SH-4 antibodies recognize distinct epitopes on CD73 from human mesenchymal stem cells”, Biochem Biophys Res Commun. 2001; 289: 519-24; and Pittenger MF, et al., “Multilineage potential of adult human mesenchymal stem cells”, Science. 1999; 284: 143-7). However, the drawback of the existing markers is that they are all nonspecific for MSCs, but still recognize other cell populations in the bone marrow.

The cell surface marker CD271 is at present the most specific cell surface marker for the isolation of mesenchymal stem cells that is commercially available. For example, monoclonal antibodies against this marker are marketed by the companies BD PharMingen, San Diego, USA, and Miltenyi Biotech, Bergisch Gladbach, Germany. However, it has been found that this marker is not selective for MSCs, but is also expressed on other CD45-positive hematopoietic cells. As a result, in a method of isolation with anti-CD271 antibodies, not only mesenchymal cells, but also hematopoietic cells are isolated.

Furthermore, the production of monoclonal antibodies is very time-consuming and expensive.

### SEQUENCE LISTING

The Sequence Listing is submitted as an ASCII text file [7291-90307-01_Sequence_Listing.txt, Dec. 5, 2012, 115 KB], which is incorporated by reference herein.

### BRIEF DESCRIPTION OF THE DRAWINGS

FIG. 1 shows a comparative analysis of the adherence of human mesenchymal stem cells (MSC; samples 5, 6, red/blue columns, right) compared with human fibroblasts (samples 1 to 4, green/yellow columns, left-middle).

FIG. 2A-2C shows measurement examples for the peptides derived from laminin, collagen-1 and collagen-4 (A), fibronectin (B) or osteopontin (C) compared with the positive control (PLL in A), with the negative control (Mal-BSA in A) or other peptides with MSC.

FIG. 3A-3C shows measurement examples for the peptides derived from laminin, collagen-1 and collagen-4 (A), fibronectin (B) or osteopontin (C) compared with the positive control (PLL in A), with the negative control (Mal-BSA in A) or other peptides with fibroblasts as controls.

### SUMMARY OF THE INVENTION

Against the background of the disadvantages known from the prior art, it is an object of the present invention to offer new possibilities for isolating mesenchymal stem cells (MSCs) that are as pure as possible, and for separating them from contaminating cells, for example fibroblasts. Fibroblasts actually express the inclusion and exclusion criteria defined by Dominici et al. (Cytotherapy, 2006; 8: 315-7), and cannot be distinguished microscopically from MSCs. However, they are certainly not differentiation-competent. Therefore separation of the MSCs from fibroblasts is very important.

According to the invention, this and other objects are solved by a method for selecting and/or enriching mesenchymal stem cells, wherein a protein or a peptide derived therefrom, which is selected from the group comprising laminin-1, collagen-1, collagen-3, collagen-4, tenascin, thrombospondin-1, osteopontin, fibronectin, vitronectin, or fragments thereof, or mixtures thereof, is/are used to bind to and to identify MSC.

According to one aspect of the invention, the protein employed in the method according to the invention has a sequence that is selected from SEQ ID No. 1 to 7, and more preferably, if the peptide derived therefrom has one of the SEQ ID Nos. 8 to 32 of the appended sequence listing.

The proteins provided or the peptides derived therefrom bind specifically and preferentially to mesenchymal stem cells, so that it is possible to isolate from a sample mesenchymal stem cells from contaminating cells such as fibroblasts for example, and use them for further applications. According to the invention, the proteins and peptide fragments presented herein can also be used for the culture and/or activation of MSCs.

The claimed method for the isolation and/or identification of mesenchymal stem cells using the mentioned proteins or peptides derived therefrom has not been described or suggested previously in the prior art.

The terms “peptides derived therefrom” or “peptide derived therefrom” or “peptide fragment” mean, in the present context, any peptide that is contained in the listed proteins, and which therefore has a continuous sequence of amino acids that is contained as such, i.e. with the same succession of amino acids, in the protein; the sequence has properties of binding to MSCs, preferably the same or similar to the whole protein. In this context it is to be understood that “protein” means in each case a whole protein identified as such with particular functions and structures, and “peptide” means a part, or a partial sequence thereof.

Laminin is a glycoprotein with a 14% carbohydrate component, mainly occurring in the basal lamina of epithelia and endothelia. The laminin molecule consists of an α-, a β- and a γ-protein chain, which are assembled in heterotrimer form into the respective laminin molecule. Currently, 15 different laminin isoforms are known.

Collagen is a water-insoluble protein of the extracellular matrix that has a fibrous structure and is one of the scleroproteins, and is involved in particular in the construction of connective tissues, e.g. skin, blood vessels, ligaments, tendons and cartilage, and in the construction of bones and teeth (dentin). At present approx. 28 different types of collagen are known (types I to XXVIII), all having in common that they are built up from three polypeptide chains, which are called collagen helices and are wound around one another in the form of a triple helix. Collagen-1 and collagen-3, along with collagen-2, -9 and -11, are among the fibrillar collagens. Collagen-1, a trimer, consists of [α1(U)₂α2(I)] (alpha-1 type I collagen) or 3 [α1(I)] chains; collagen-3 is a homotrimer of 3 [α1(III)] chains.

Finally, tenascin is an oligomeric glycoprotein of the extracellular matrix, which takes part in interactions between epithelial and mesenchymal cells. In vertebrates, so far 3 different types of tenascin have been described (tenascin-R, -C and -X), which differ inter alia in the number of certain domains, namely the EGF (epidermal growth factor) and fibronectin-type-III-like domains. Tenascin is involved in the regeneration of nerve tissue in the adult organism. In adult skin, tenascin is induced during wound healing. Tenascin directs the migration of cells in these processes, and can stimulate or inhibit cell adhesion via various protein domains.

Osteopontin is a glycoprotein in all higher mammals, and is involved in the maintenance of bone substance and some immune processes. It binds hydroxyapatite and forms the basic structure for bones. Synonyms of the protein are sialoprotein 1 and 44K BPP (bone phosphoprotein).

Thrombospondin-1 belongs to a family of proteins that take part in various biological processes. The protein family consists of thrombospondin-1 to −5, divided into subgroups: subgroup A consists of TSP1 and TSP2, which are homotrimers; subgroup B consists of TSP3, TSP4 and TSP5. TSP1 is involved in many different biological processes, such as angiogenesis, apoptosis, activation of the tissue hormone TGF and immune regulation.

Fibronectin (from the Latin: fibra for “fiber”; nexus for “linkage”) is an extracellular glycoprotein that plays an important role in many physiological processes. It is a heterodimer of two rod-shaped polypeptide chains, which are held together by disulfide bridges near the C-terminal end. So far more than 20 different isoforms have been found, which are produced by alternative splicing of the mRNA of a single gene. An individual fibronectin polypeptide chain (˜230 kDa) consists of a large number of domains (approx. 40-90 amino acids), which are divided into structure types I, II, and III based on their homology.

Vitronectin is a glycoprotein that occurs in serum and the extracellular matrix, and is a secreted protein, which is either in the form of an individual chain, or a double chain joined together by a disulfide bridge. The reference number for this protein in the NCBI (National Center for Biotechnology Information) database (GenBank®) is NP_{—}000629).

According to one aspect of the invention, the protein or the peptide derived therefrom has a sequence that is selected from SEQ ID No. 1 to 32 of the appended sequence listing, or that of a binding-relevant sequence contained therein.

The sequence with ID No. 1 is the sequence of the alpha-1 chain of human laminin-1 and is for example listed in GenBank® of the National Center for Biotechnology Information under number NP_{—}005550; the sequence with ID No. 2 is the sequence of the beta-1 chain of human laminin-1 (GenBank® No. NP_{—}002282), the sequence with ID No. 3 is the sequence of the gamma-1 chain of human laminin-1 (GenBank® No. NP_{—}00284).

Similarly, the sequence shown as SEQ ID No. 4 is the sequence of the alpha-1(I) chain of human collagen (GenBank® No. P02452), the sequence with ID No. 5 is the sequence of the alpha-2(I) chain of human collagen (GenBank® No. P08123), and the sequence with ID No. 6 is the sequence of the alpha-1-(III) chain of human collagen (GenBank® No. P02461). The three sequences with SEQ ID No. 4, 5 and 6 therefore represent sequences for the trimer collagen-1. The sequence with SEQ ID No. 6 is, moreover, also a component of the homotrimer collagen-3.

The sequence with ID No. 7 is the sequence of human tenascin-C, and has the identification CAA55309 in GenBank®.

According to another aspect of the invention, one of the following binding-specific peptide fragments is used in the use according to the invention:
a) a peptide that has one of the SEQ ID numbers SEQ ID No. 8 to 32 given in the sequence listing, or
b) fragments of the sequences according to a), which have a substantially identical biological activity of the peptide according to a) in an assay relating to the binding of mesenchymal stem cells,
c) a peptide fragment with a sequence that is identical to at least 80%, preferably to at least between 80% and 99%, to one of the sequences stated in a) and b).

The sequences with SEQ ID Nos. 8 to 32 are preferred peptides, to which mesenchymal stem cells can be bound specifically. The peptides with SEQ ID Nos. 8 (GF-Orn-GER; contains ornithine at position 3) and 9 (GEFYFDLRLKGDK) are derived from collagen-1 and -4, SEQ ID Nos. 10 to 13 from laminin (LRE, laminin; AASIKAVAVSADR, laminin alpha-1 chain; LAIKNDNLVYVY, DVISLYNFKHIY (SEQ ID No. 23), in each case laminin alpha-4 chain; RYVVLPRPVLFEK, laminin beta-1 chain), SEQ ID Nos. 14 and 15 from thrombospondin (ELTGAARKGSGRRLVKGPD, thrombospondin-1; MKKTRGTLLALERKDHS, thrombospondin-1), SEQ ID No. 16 from osteopontin (SVVYGLR), and SEQ ID Nos. 17 to 20 and 32 from fibronectin (YIIR; GSKS; TYSSPEDGIHE; WQPPRARITGY; DELPQLVTLPHPNLHGPEILDVPST).

It will be clear to a person skilled in the art that in addition to the aforementioned proteins/peptides, for the purpose of the method according to the invention it is also possible to use proteins/peptides that are, for example owing to sequence homologies, functionally identical to the disclosed peptides, so that for example it is also possible to use proteins that may possibly be deletions, substitutions, insertions, etc. compared with the aforementioned proteins/peptides, but which nevertheless have the same function as the stated proteins/peptides. Thus, for example, it is also possible to use just binding-relevant fragments of the stated proteins/peptides.

In this context, “a substantially identical biological activity (of a peptide) in an assay relating to the binding of mesenchymal stem cells” means that variants or derivatives of the peptides with SEQ ID Nos. 8 to 32 are also suitable in the context of the present invention for the method according to the invention, with which a similarly effective and specific binding of mesenchymal stem cells is achieved. It is to be understood in this context that “substantially” means an activity that is almost identical to the values for the peptides disclosed concretely with their sequences. It will be clear to a person skilled in the art or may become apparent with reasonable, simple tests on the basis of the disclosed sequences, what further sequence variants are possible in order to achieve said similar binding specificity and efficacy.

The term “hybridization under stringent conditions” means in the context of this invention that the hybridization is carried out in vitro under conditions that are sufficiently stringent to ensure a specific hybridization. The term “specific hybridization” relates to the circumstance that a molecule binds under stringent conditions preferentially to a particular nucleic acid sequence, the target sequence, when this is part of a complex mixture of e.g. DNA or RNA molecules, but not or at least to a considerably reduced extent to other sequences. The precise conditions for stringency depend on the corresponding circumstances, for example with respect to the material used. Typically, stringent conditions are those for which hybridization takes place between the stated nucleotide sequences under usual conditions, especially at 20° C. below the melting point of the stated nucleotide sequences. Preferred hybridization conditions are for example those for which a solution of 5× or 6×SSPE (or SSC), 1% or 0.5% SDS, 1×Denhardt's solution is used, and the hybridization temperatures are between 35° C. and 70° C., preferably at 65° C. Hybridization is preferably followed by washing first with 2×SSC, 1% SDS and then with 0.2×/0.1×SSC at temperatures between 35° C. and 70° C., preferably at 65° C. (for definitions of SSPE, SSC and Denhardt's solution see Sambrook et al., Molecular Cloning: A Laboratory Manual, 2nd edition, Cold Spring Harbor Laboratory Press, Cold Spring Harbor N.Y. (1989)).

The term “sequence homology” or “sequence identity” denotes the proportion of bases coinciding between two nucleic acid sequences or the proportion of amino acids coinciding between two amino acid sequences. When the sequence homology is expressed as a percentage, e.g. 90%, the percentage denotes the proportion of agreement over the length of the sequence, which is compared with another sequence.

It is further to be understood that on the one hand proteins/peptides derived therefrom of human origin can be used, but that on the other hand functionally and structurally similar/identical proteins from other mammals can also be used, as well as artificially synthesized or recombinantly produced peptides/proteins, which have the sequence of the stated proteins/peptides, or parts thereof.

With the aforementioned peptides it could be shown that specific binding of mesenchymal stem cells to the peptides is possible.

According to an embodiment of the method of the invention, e.g., the bottom of a culture flask is coated with the proteins or the peptides derived therefrom and, thus, it is possible to keep the MSCs binding thereto in the undifferentiated state, so that larger batches of differentiation-competent MSCs can be produced.

On the other hand, and according to another aspect, the proteins or the peptides derived therefrom can be employed for the direct isolation and enrichment of MSCs from primary tissue, for example from bone marrow, umbilical cord blood, etc.

According to another aspect, the proteins or the peptides derived therefrom are employed for modifying or stabilizing the specific differentiation of the mesenchymal stem cells into the mature mesenchymal cell types, such as chondrocytes, osteoblasts, adipocytes, myocytes.

According to yet another embodiment, the proteins or the peptides derived therefrom are also employed in in-situ applications: for example, supporting structures such as implants or stents etc. are to be coated with the peptides. The MSCs bind via these peptides to the supporting structures, so that the MSCs are concentrated or enriched locally on the carrier materials. Through differentiation of these concentrated mesenchymal stem cells into the corresponding tissue, for example injured or damaged tissue can be regenerated or new tissue can be formed at the site of the carrier structure. Advantageously, the peptides are of human origin, so that no immune reactions occur.

According to the invention, the proteins or the peptides derived therefrom are used in a method for selecting MSCs from a sample that contains MSCs and other cell types.

For example, as already mentioned above, by using the disclosed proteins or the peptides derived therefrom, MSCs can be isolated directly from a primary culture, or else from a previously cultivated cell population.

Moreover, in another embodiment of the method according to the invention, the peptide can be used for labeling of MSCs.

In this embodiment, the protein to be used or the peptide derived therefrom can be modified or labeled, for example with fluorescent groups, so that after binding of the protein/peptide to the MSCs these can be identified simply and quickly on the basis of the labeling.

According to the invention, the protein or the peptide derived therefrom is used for enrichment of MSCs. Enrichment can, for example, be achieved in vitro or in situ, for example by coating suitable materials/structures with the peptides, as mentioned above.

Moreover, according to another embodiment, the protein or the peptide derived therefrom is used in a method for inducing the proliferation or differentiation of MSCs.

The proteins or the peptides derived therefrom can in this embodiment be used specifically for modifying the differentiation of the MSCs for example into osteoblasts, chondrocytes, adipocytes, etc.

According to another aspect of the invention, a method for treating wounds, injuries and/or degenerated tissue is disclosed, wherein a protein or a peptide derived therefrom is administered to a patient, which protein/peptide derived therefrom is selected from the group comprising laminin-1, collagen-1, collagen-3, collagen-4, tenascin, thrombospondin-1, osteopontin, fibronectin, or binding-specific fragments or mixtures thereof.

With the new method, it is possible to employ protein/peptide structures that are of human origin, and therefore cause little if any immune reaction, when they are introduced into a patient's body, for example in conjunction with a carrier structure on which they are immobilized.

The tissue to be treated can be any bone, cartilage and/or muscle tissue, preferably human.

It is especially preferable if the medicinal product is an implant, especially a stent, coated with at least one peptide.

A stent is an endoprosthesis, of various materials, which generally serve for maintaining patency of vessels in the body, and are of tubular design and/or as a meshwork with or without a sheath. Stents are introduced in compressed form by means of a suitable insertion system into the vessels and at the destination are deployed to remain there. In the use according to the invention it is intended that the stent to be used is coated with the protein or the peptide derived therefrom and then introduced into the vessel to be treated.

On the other hand, implants, for example for skin, cartilage or bone replacement or regeneration, can be coated with the peptides and implanted in the region of the body to be treated. For example, implants for treating knee and disk injuries can be coated, and it will be clear to a person skilled in the art that any other implant that is implanted in a body for the regeneration or replacement of tissue can be correspondingly coated. After implantation in the place to be treated in a patient's body, the MSCs are concentrated/enriched there by means of the peptides present on the carrier, so that a kind of “sticking plaster” is created.

Accordingly, the invention also relates to a method for the production of an implant, wherein a protein or a peptide derived therefrom is employed, which is selected from the group comprising laminin-1, collagen-1, collagen-3, collagen-4, tenascin, thrombospondin-1, osteopontin, fibronectin, or binding-specific fragments or mixtures thereof.

The invention, thus, relates to a method of isolating, identifying, cultivating and activating mesenchymal stem cells, comprising the step of selecting the stem cells from a sample containing stem cells with a protein or peptide derived therefrom, which is selected from laminin-1, collagen-1, collagen-3, collagen-4, tenascin, thrombospondin-1, osteopontin, fibronectin, or binding-specific fragments or mixtures thereof, especially with a peptide of SEQ ID No. 1 to 32.

The stem cells that are obtained with this method according to the invention, which are also covered by this invention, can in their turn, as mentioned above, be employed in a method for the treatment of, for example, degenerated or injured tissue, for example for treating bone, cartilage and/or muscles.

Therefore the present invention also relates a method for the production of a medicinal product, and in particular for treating wounds, injuries and/or degenerated tissue, especially cartilage, bone, muscles, vessels, or for immuntherapy, or as trophic cells, or as a vehicle for recombinant (gene) therapy, wherein mesenchymal stem cells are employed, which mesenchymal stem cells have been isolated, identified, cultivated or activated according to one of the methods according to the invention, or that have been enriched according to the invention, wherein they need not then necessarily be detectable in the repair tissue.

The invention further relates to a synthetic, isolated or recombinant peptide, comprising an amino acid sequence selected from the group consisting of:
a) the amino acid sequence according to SEQ ID No. 8 to 32,
b) fragments of the sequences according to a), which possess the biological activity of the peptide according to a) in an assay relating to the binding of mesenchymal stem cells,
c) fragments with a sequence that is identical to at least 80%, preferably to at least between 80% and 99%, to one of the sequences stated in a) and b).

As shown in the inventors' experiments, these peptides bind mesenchymal stem cells specifically. Therefore the peptides provide a means for isolating and/or enriching mesenchymal stem cells.

The peptides can also be expressed using genetic engineering techniques and expressed as structures, both membrane-bound on cells, and in dissolved form and in combination with other proteins (e.g. as peptide+collagen-1 fibers for augmented biomaterial).

In particular, the peptides according to the invention can thus also be used directly for treating wounds, injuries and/or degenerated tissue.

Therefore the invention also relates to pharmaceutical compositions that have at least one of the peptides according to the invention and a pharmaceutically acceptable carrier.

“Pharmaceutically acceptable carrier” means in the present context a nontoxic material that does not impair the efficacy of the biological activity of the active substance in the composition.

It is also to be understood that in another embodiment the pharmaceutical composition contains other therapeutically and/or pharmaceutically active ingredients, which—depending on the disease(s) to be treated—are additionally administered in the pharmaceutical composition.

The disease to be treated is preferably a disease of humans.

The pharmaceutical composition can be administered systemically, i.e. for example by the oral, subcutaneous, intravenous, rectal, parenteral, intramuscular, interperitoneal, transdermal, or topical route, wherein the method of administration will depend on the type of disease, the clinical picture, and the patient's condition. Moreover, administration can take place once or can be repeated, with administration taking place in the latter case once or several times daily, and/or over a longer period.

In addition to the active substances, the pharmaceutical composition can also contain buffers, diluents and/or additives. Suitable buffers include for example Tris-HCl, glycine and phosphate, and suitable diluents for example aqueous NaCl solutions, lactose or mannitol. Suitable additives include for example detergents, solvents, antioxidants and preservatives. A review of these additional ingredients is given for example in A. Kibbe.: “Handbook of Pharmaceutical Excipients”, 3rd Ed., 2000, American Pharmaceutical Association and Pharmaceutical Press.

Further advantages and features can be seen from the following description and the appended drawing.

It is to be understood that the features mentioned above and other features yet to be explained below can be applied not only in the combination stated in each case, but also in other combinations or on their own, while remaining within the scope of the present invention.

A practical example of the invention is shown in the drawing and will be described in more detail below.

### DESCRIPTION OF PREFERRED EMBODIMENTS

### Example 1

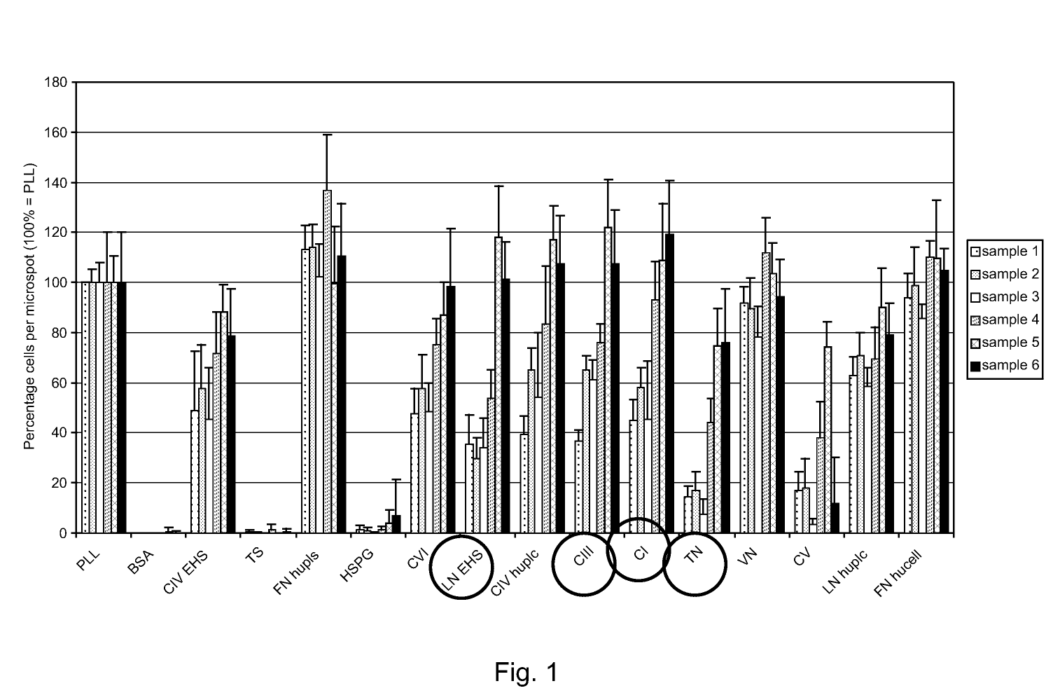

The adherence of human mesenchymal stem cells (MSC, samples 5 and 6 in FIG. 1) and human fibroblasts (samples 1 to 4 in FIG. 1) on matrix proteins was investigated in a comparative test. The investigation was carried out using a chip (Multiple Substrate Array (MSA®)), on which the proteins were immobilized. The procedure used as such was as described in Kuschel et al., “Cell adhesion profiling using extracellular matrix protein microarrays”, Biotechniques 40: 523-531 (2006).

MSCs were enriched from bone marrow by density gradient centrifugation, cultivated as an adherent population in MSC-expansion medium, and harvested. The differentiation potential was tested on an aliquot and adipogenic, chondrogenic and osteogenic differentiation was confirmed. Therefore the cells fulfill the physiological requirements for MSCs (M. Dominici et al. 2006, Cytotherapy, 8: 315 ff). It is to be understood that MSCs from other tissues can also be used, for example fat tissue or any other tissue in which there are MSCs.

Fibroblasts were isolated from the synovial membrane and expanded as described (Aicher et al. 1994 J. Immunol. 152: 5940 ff). The cells were harvested, washed, counted and in each case 20 000/test chamber were investigated using MSA™ chip technology for adherence to proteins. Briefly, the cells were in each case incubated on a microarray of 12 different proteins in a chamber first for uniform distribution with shaking (two hours), and then without shaking (two hours) under usual culture conditions. After this incubation, non-adherent cells were washed away by rinsing the arrays. Cells adhering to the proteins were fixed chemically and their nuclei were marked with dye, for quantitation of the number of adherent cells per microspot of protein. Instead of the fibroblasts used here, it is also possible for example to use skin fibroblasts, chondrocytes, osteoblasts, meniscus cells, or other cells.

The results of this experiment are shown in FIG. 1, wherein the adherence on the proteins laminin-1 (LN EHS), collagen-3 (CIII) and collagen-1 (CI) and tenascin-C (TN) is shown with the eighth, tenth, eleventh or twelfth protein in the diagram in FIG. 1. The other proteins tested were: fibronectin (FN hulps), collagen-6 (CVI), vitronectin (VN), collagen-4 (CIV EHS), and laminin-10 (LN huplc).

The protein poly-L-lysine (PLL) served as positive control, bovine serum albumin (BSA) as negative control. Both populations, i.e. MSCs and fibroblasts, furthermore, do not bind to thrombospondin.

It can be seen from the results shown in FIG. 1 that the binding of the MSCs to laminin-1, collagen-1 and collagen-3 and tenascin-C was firmer than the binding of fibroblasts to these proteins. Therefore these proteins are especially suitable for the present uses according to the invention.

### Example 2

The adhesion of cells to peptides was also measured using the MSA™ technology (see Kuschel et al., 2006) as above in example 1. Instead of the extracellular matrix proteins, however, peptides coupled to bovine serum albumin were printed in the form of microarrays (8×8 microspots) onto the nitrocellulose layer, and then the remaining surface was sealed. For incubation with cells, small silicone chambers were placed on the coated slides, and incubated with MSCs or fibroblasts. After incubation for two hours, the non-adherent cells were washed away.

Adherent cells were made visible on the white nitrocellulose film by staining with Coomassie Blue and were evaluated in a motorized photomicroscope: the number of blue cells or the color intensity per spot is measured and transferred to tables for evaluation.

Evaluation of these arrays using the semi-automatic photomicroscope gave the relative binding strength on PLL (standardized to 100%, positive control) and BSA (standardized to 0%, negative control) for the peptides tested in each case.

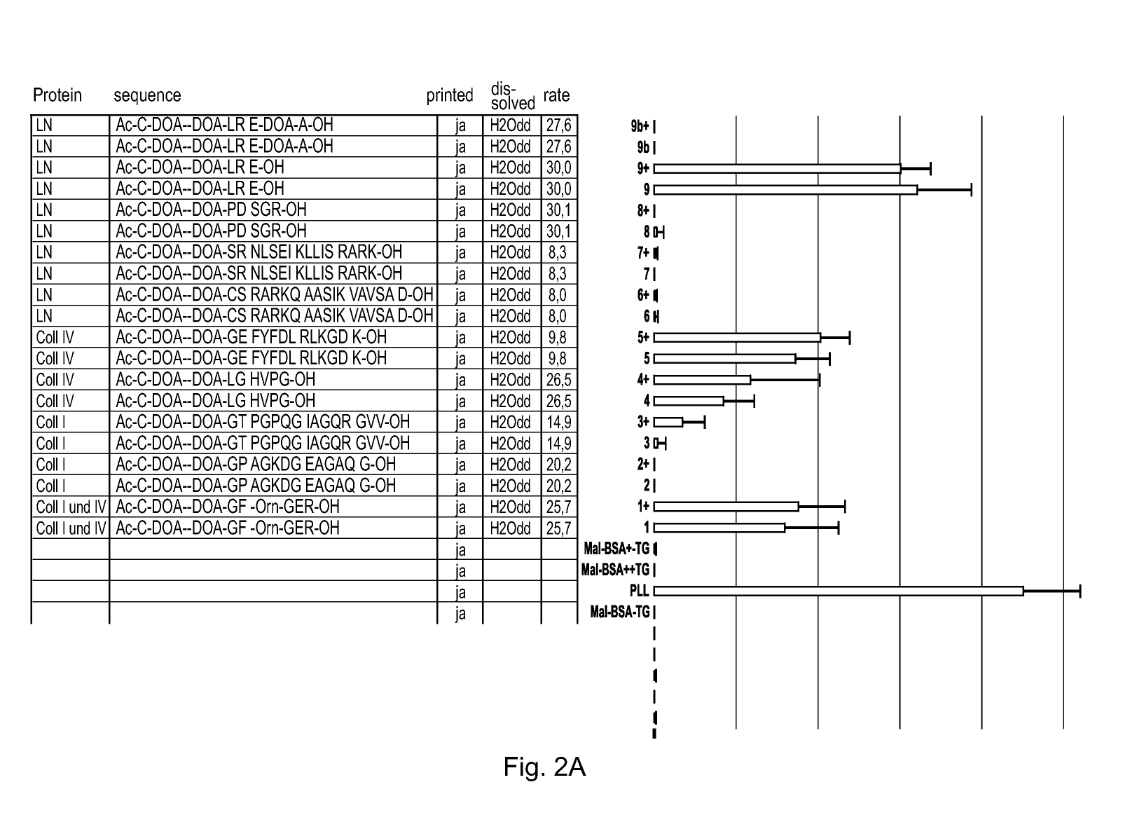

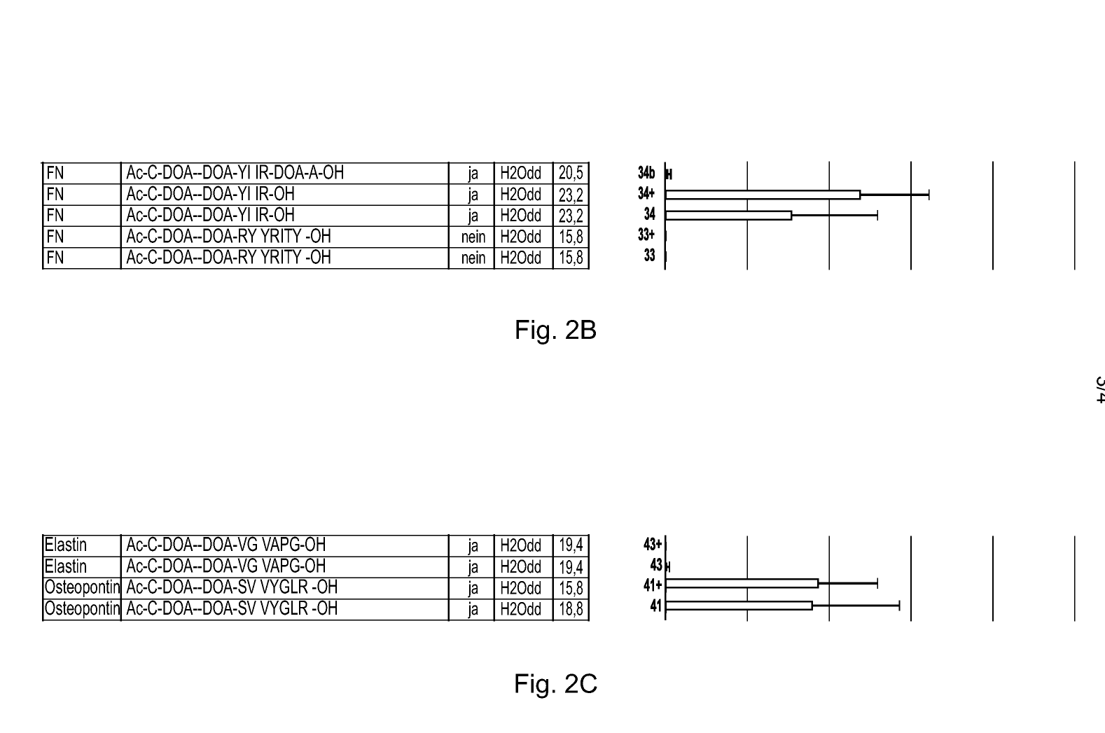

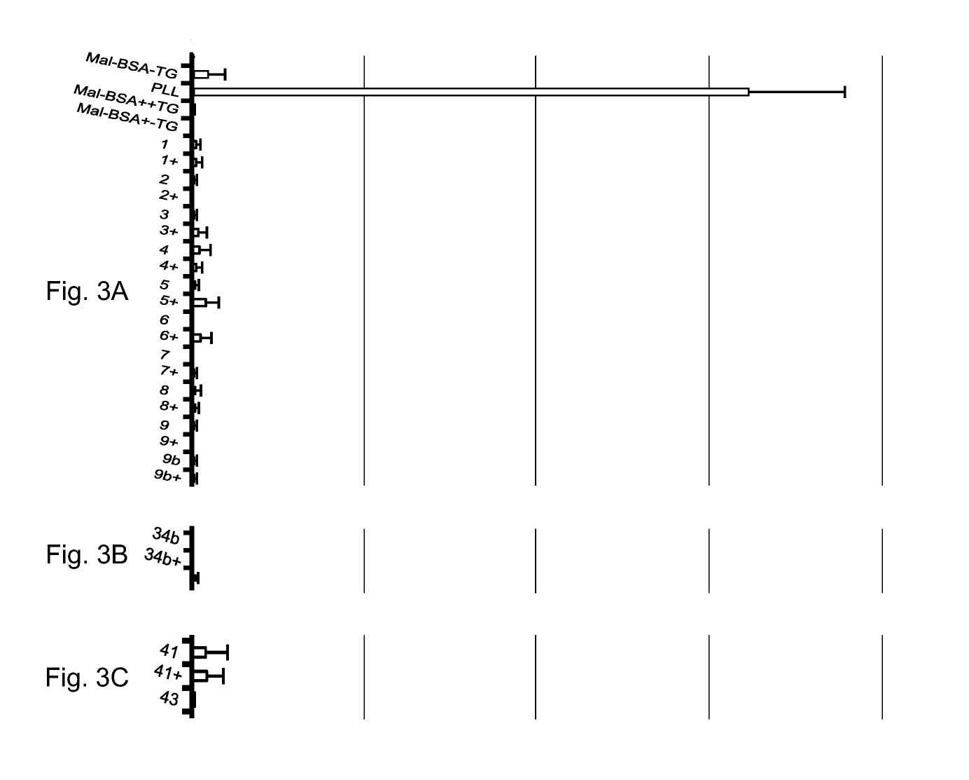

FIG. 2 shows the results achieved with different peptides and MSCs. The results presented in FIG. 2 show that laminin-derived peptides No. 9, #9-COOH (SEQ ID No. 10), No. 15 and No. 16, the collagen-derived peptides No. 1 and No. 5 (SEQ ID Nos. 8 and 9), the vitronectin-derived peptide No. 37b, and the fibronectin-derived peptides No. 11, No. 20, No. 21, No. 21-COOH, No. 22, No. 22-COOH, No. 34, No. 34-COOH, and No. 30 make the adhesion of MSCs possible (FIG. 2), but not that of fibroblasts (FIG. 3). The sequences of the MSC-binding peptides are shown in table 1:

TABLE 1

| No. | Sequence | SEQ ID No. |
|---|---|---|
| 1 | GF-Orn-GER-OH | 8 |
| 5 | GEFYFDLRLKGDK-OH | 9 |
| 9 | LRE-OH | 10 |
| 9-COOH | LRE-Doa-A-OH | 21 |
| 11 | GRGDSO-OH | 22 |
| 15 | LAIKNDNLVYVY-OH | 12 |
| 16 | DVISLYNFKHIY-OH | 23 |
| 20 | DRVPHSRNSIT-OH | 24 |
| 21 | KREDVY-OH | 25 |
| 21-COOH | KREDVY-COOH | 26 |
| 22 | EILDV-OH | 27 |
| 22-COOH | EILDV-COOH | 28 |
| 30 | WTPPRAQITGYRLTVGLTRR-OH | 29 |
| 34 | YIIR-OH | 17 |
| 34-COOH | YIIR-DOA-A-OH | 30 |
| 37b | KRSR-DOA-A-OH | 31 |
| 41 | SVVYGLR-OH | 16 |

What is claimed is:

1. A method for selecting and/or enriching mesenchymal stem cells (MSCs) from a sample, comprising the steps of
contacting a sample containing mesenchymal stem cells and other cell types with a peptide, or a fragment thereof, wherein the peptide or fragment thereof specifically binds to mesenchymal stem cells, and wherein the peptide is selected from the group consisting of laminin-1, collagen-1, collagen-3, collagen-4, tenascin, thrombospondin-1, osteopontin, fibronectin, vitronectin, and mixtures thereof, thereby allowing binding of the peptide or fragment thereof to mesenchymal stem cells,
selecting and/or enriching the mesenchymal stem cells bound to the peptide or fragment thereof from the other cell types not bound to the peptide or fragment thereof.

2. The method as claimed in claim 1, wherein the peptide fragment is:
a) a peptide consisting of the amino acid sequence set forth as one of SEQ ID NOs: 8 to 32;
b) fragments of the sequences according to a), which have a substantially identical biological activity of the peptide according to a) in an assay relating to the binding of mesenchymal stem cells; or
c) a peptide fragment with a sequence that is at least 80% identical one of the sequences stated in a) and b).

3. The method as claimed in claim 1, wherein the sample is from a primary culture or a previously cultured cell population.

4. The method as claimed in claim 1, wherein the peptide or fragment thereof is labelled.

5. The method as claimed in claim 1, wherein the selecting and/or enriching step comprises identifying and/or isolating the MSCs bound to the peptide or fragment thereof.

6. The method as claimed in claim 1, wherein the step of selecting and/or enriching is in vivo, in situ or in vitro.

7. The method as claimed in claim 1, further comprising the step of inducing proliferation and/or differentiation of the MSCs bound to the peptide or fragment thereof, wherein the proliferation and/or differentiation is induced by the peptide and/or fragment thereof.

8. A method for treating wounds, injuries and/or degenerated bone, cartilage or tissues comprising administering to a patient in need thereof a peptide consisting of one of the amino acid sequences set forth as SEQ ID NOs: 8 to 32, or mixtures thereof, thereby treating the wounds, injuries and/or degenerated bone, cartilage or tissues.

9. The method as claimed in claim 8, comprising administering to the subject an implant or stent coated with the peptide.

10. A method for the production of an implant, the method comprising the step of
coating the implant with a peptide consisting of the amino acid sequence set forth as one of SEQ ID NOs: 8 to 32, or mixtures thereof, thereby producing the implant.

11. Method for treating wounds, injuries and/or degenerated bone, cartilage or tissues comprising administering to a patient in need thereof a mesenchymal stem cell that has been selected or enriched according to the method as claimed in claim 1.

12. Synthetic, isolated or recombinant peptide, consisting of an amino acid sequence selected from the group consisting of:
a) the amino acid sequence set forth as one of SEQ ID NOs: 8 to 3;
b) fragments of the sequences according to a), which possess the biological activity of the peptide according to a) in an assay relating to the binding of mesenchymal stem cells; and
c) fragments with a sequence that is identical to at least 80%- to one of the sequences stated in a) and b).

13. A pharmaceutical composition comprising at least a) one peptide as claimed in claim 12 and b) a pharmaceutically acceptable carrier.
